(19) United States
(12) Patent Application Publication
(10) Pub. No.: US 2023/0113096 A1
(43) Pub. Date: Apr. 13, 2023

(54) NONLINEAR OPTICAL MATERIAL, LIGHT ABSORBING MATERIAL, RECORDING MEDIUM, METHOD FOR RECORDING INFORMATION, AND METHOD FOR READING OUT INFORMATION

(71) Applicant: Panasonic Intellectual Property Management Co., Ltd., Osaka (JP)

(72) Inventors: NAOYA SAKATA, Hyogo (JP); KENJI TAGASHIRA, Osaka (JP); MASAKO YOKOYAMA, Osaka (JP); KOTA ANDO, Osaka (JP)

(21) Appl. No.: 18/051,555

(22) Filed: Nov. 1, 2022

Related U.S. Application Data
(63) Continuation of application No. PCT/JP2021/016253, filed on Apr. 22, 2021.

(30) Foreign Application Priority Data
May 22, 2020 (JP) 2020-090123

(51) Int. Cl. G11B 7/2467; G02F 1/35; G02F 1/361; G11B 7/24065

(52) U.S. Cl. CPC G11B 7/2467; G02F 1/3523; G02F 1/3611; G11B 7/24065; G11B 2007/24624

(57) ABSTRACT

A nonlinear optical material is represented by the following formula (1). At least one selected from the group consisting of R¹ to R⁵, at least one selected from the group consisting of R⁶ to R¹⁰, and at least one selected from the group consisting of R¹¹ to R¹⁵ are represented by the following formula (2).

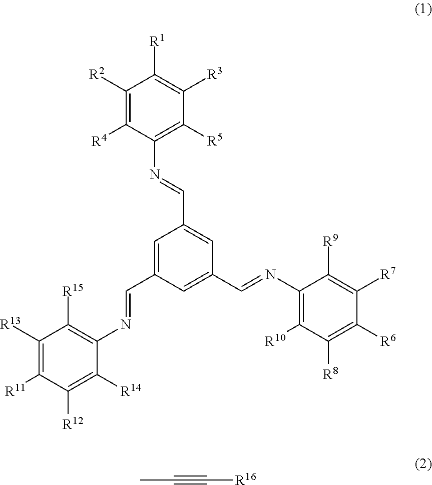

### BACKGROUND

### 1. Technical Field

The present disclosure relates to a nonlinear optical material, a light absorbing material, a recording medium, a method for recording information, and a method for reading out information.

### 2. Description of the Related Art

Among optical materials such as a light absorbing material, a material having a nonlinear optical effect is called a nonlinear optical material. The nonlinear optical effect means that when a substance is irradiated with strong light, such as laser light, an optical phenomenon proportional to the square or higher order of the electric field of the irradiation light occurs in the substance. Examples of the optical phenomenon include absorption, reflection, scattering, and luminescence. Examples of a secondary nonlinear optical effect that is proportional to the square of the electric field of irradiation light include second harmonic generation (SHG), Pockels effect, and parametric effect. Examples of a tertiary nonlinear optical effect that is proportional to the cube of the electric field of irradiation light include two-photon absorption, multiphoton absorption, third harmonic generation (THG), and Kerr effect.

Various studies have been actively carried out on nonlinear optical materials. In particular, as nonlinear optical materials, inorganic materials that can be easily prepared into a single crystal have been developed. In recent years, a nonlinear optical material made of an organic material is expected to be developed. Examples of the nonlinear optical material made of an organic material include organic dyes. Organic material not only have high degrees of freedom in design but also have large nonlinear optical constants, compared to inorganic materials. Furthermore, in organic materials, nonlinear responses are performed at high speeds. In the present specification, a nonlinear optical material including an organic material may be called an organic nonlinear optical material.

### SUMMARY

In one general aspect, the techniques disclosed here feature a nonlinear optical material represented by the following formula (1):

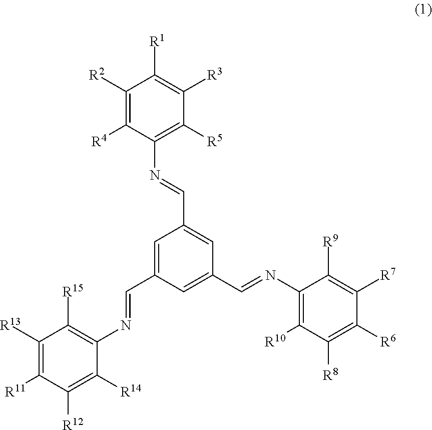

in the formula (1), R¹ to R¹⁵ each independently include at least one atom selected from the group consisting of H, C, N, O, F, P, S, Cl, I, and Br. At least one selected from the group consisting of the R¹ to the R⁵, at least one selected from the group consisting of the R⁶ to the R¹⁰, and at least one selected from the group consisting of the R¹¹ to the R¹⁵ are represented by the following formula (2):

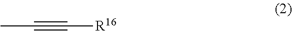

in the formula (2), R¹⁶ includes at least one atom selected from the group consisting of H, C, N, O, F, P, S, Cl, I, and Br.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a flow chart relating to a method for recording information using a recording medium including a compound according to an embodiment of the present disclosure;

FIG. 1B is a flow chart relating to a method for reading out information using a recording medium including a compound according to an embodiment of the present disclosure;

FIG. 2 is a graph showing a ¹H-NMR spectrum of the compound of Example 1; and

FIG. 3 is a graph showing a ¹H-NMR spectrum of the compound of Example 2.

### DETAILED DESCRIPTION

### Underlying Knowledge Forming Basis of the Present Disclosure

Among organic nonlinear optical materials, two-photon absorption materials are receiving particular attention. Two-photon absorption is a phenomenon in which a compound absorbs two photons almost at the same time and transits to an excited state. As the two-photon absorption, non-resonance two-photon absorption and resonance two-photon absorption are known. The non-resonance two-photon absorption means two-photon absorption in a wavelength region where there is no one-photon absorption band. In the non-resonance two-photon absorption, a compound absorbs two photons almost at the same time and transits to a higher excited state. In the resonance two-photon absorption, a compound transits to a higher excited state by absorbing a first photon and then further absorbing a second photon. In the resonance two-photon absorption, the compound successively absorbs two photons.

In the non-resonance two-photon absorption, the light amount absorbed by a compound is usually proportional to the square of irradiation light intensity. Accordingly, for example, regarding light collected by a lens, two-photon absorption by a compound can be caused at only near the focal point where the light intensity is high. That is, in a sample including a two-photon absorption material, it is possible to excite the compound at a desired position only. Thus, since compounds causing non-resonance two-photon absorption provide extremely high spatial resolution, application to uses, such as a recording layer of a three-dimensional optical memory and a photocurable resin composition for stereolithography, is being studied.

The study of two-photon absorption materials that are used in, for example, a recording layer of a three-dimensional optical memory or a photocurable resin composition for stereolithography is actively being carried out. In a two-photon absorption material, as an indicator showing the efficiency of two-photon absorption, a two-photon absorption cross section (GM value) is used. The unit of the two-photon absorption cross section is GM (10⁻⁵⁰ cm⁴·s·molecule⁻¹·photon⁻¹). A large number of compounds having a two-photon absorption cross section large enough to exceed 500 GM have been reported (for example, Harry L. Anderson et al, “Two-Photon Absorption and the Design of Two-Photon Dyes”, Angew. Chem. Int. Ed. 2009, Vol. 48, p. 3244-3266). However, in most of the reports, the two-photon absorption cross section is measured using laser light having a wavelength of longer than 600 nm. In particular, in some of them, as the laser light, near-infrared light having a wavelength of longer than 750 nm is used.

However, in order to apply a two-photon absorption material to industrial uses, the material is required to have a large two-photon absorption cross section when irradiated with laser light having a shorter wavelength. For example, in the field of optical memory, from the viewpoint of diffraction limit of collected laser light, laser light having a short wavelength is used for realizing a finer light collected spot. In a three-dimensional optical memory with a multilayer structure, the recording density can be dramatically improved by using a two-photon absorption material having extremely high spatial resolution. In particular, in the use of a three-dimensional optical memory, laser light having a central wavelength of 405 nm is used in the standard of Blu-ray® disk. Accordingly, development of a compound having a large two-photon absorption cross section for light of the same wavelength region as that of this laser light can highly contribute to industrial development.

Japanese Patent No. 5769151 discloses a compound having a large two-photon absorption cross section for light having a wavelength near 405 nm. This compound has an enlarged π electron conjugated system and a high symmetric property. Consequently, in this compound, a two-photon absorption cross section of about 23000 GM is achieved. However, this compound has not yet been put into practical use at this stage and is still in the research stage. Furthermore, in the two-photon absorption cross section of this compound has a room for improvement. For example, when a compound having a small two-photon absorption cross section is used in a three-dimensional optical memory, it is necessary to improve the intensity of laser light in some cases. Accordingly, from the viewpoint of further improving the industrial applicability, a compound having a larger two-photon absorption cross section for light having a wavelength near 405 nm.

The present inventors have newly found as a result of diligent study that a compound represented by the formula (1) described later has excellent two-photon absorption properties for light having a wavelength in a short wavelength region. In the present specification, the short wavelength region is a wavelength region including 405 nm, for example, a wavelength region of 390 nm or more and 420 nm or less. In particular, the compound represented by the formula (1) has a large two-photon absorption cross section for light having a wavelength near 405 nm.

### Brief Overview of an Aspect According to the Present Disclosure

The nonlinear optical material according to a 1st aspect of the present disclosure is represented by the following formula (1):

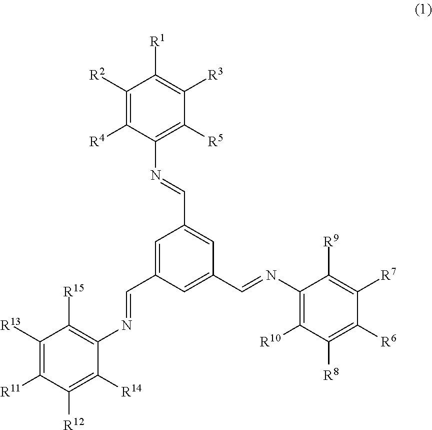

in the formula (1), R¹ to R¹⁵ each independently include at least one atom selected from the group consisting of H, C, N, O, F, P, S, Cl, I, and Br. At least one selected from the group consisting of the R¹ to the R⁵, at least one selected from the group consisting of the R⁶ to the R¹⁰, and at least one selected from the group consisting of the R¹¹ to the R¹⁵ are represented by the following formula (2):

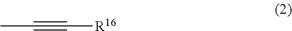

in the formula (2), R¹⁶ includes at least one atom selected from the group consisting of H, C, N, O, F, P, S, Cl, I, and Br.

According to the 1st aspect, the nonlinear optical material has excellent two-photon absorption properties for light having a wavelength in a short wavelength region.

In a 2nd aspect of the present disclosure, for example, the nonlinear optical material according to the 1st aspect may be represented by the following formula (3):

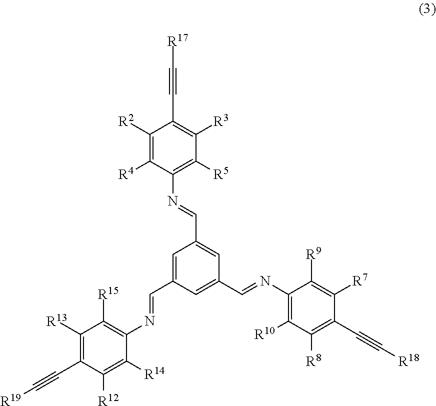

in the formula (3), R² to R⁵, R⁷ to R¹⁰, R¹² to R¹⁵, and R¹⁷ to R¹⁹ each independently include at least one atom selected from the group consisting of H, C, N, O, F, P, S, Cl, I, and Br.

In a 3rd aspect of the present disclosure, for example, in the nonlinear optical material according to the 2nd aspect, the R² to the R⁵, the R⁷ to the R¹⁰, the R¹² to the R¹⁵, and the R¹⁷ to the R¹⁹ may be each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

In a 4th aspect of the present disclosure, for example, the nonlinear optical material according to any one of the 1st to the 3rd aspects may be represented by the following formula (4):

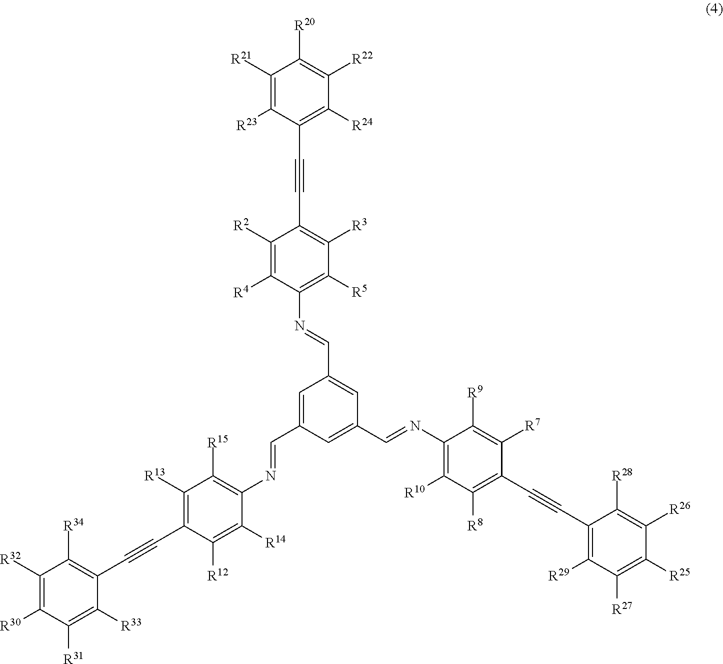

in the formula (4), R² to R⁵, R⁷ to R¹⁰, R¹² to R¹⁵, and R²⁰ to R³⁴ each independently include at least one atom selected from the group consisting of H, C, N, O, F, P, S, Cl, I, and Br.

In a 5th aspect of the present disclosure, for example, in the nonlinear optical material according to the 4th aspect, the R² to the R⁵, the R⁷ to the R¹⁰, the R¹² to the R¹⁵, and the R²⁰ to the R³⁴ may be each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

In a 6th aspect of the present disclosure, for example, the nonlinear optical material according to any one of the 1st to the 5th aspects may be used in a device that uses light having a wavelength of 390 nm or more and 420 nm or less.

According to the 2nd to the 6th aspects, the nonlinear optical material has excellent two-photon absorption properties for light having a wavelength in a short wavelength region. This nonlinear optical material is suitable for use in a device using light having a wavelength of 390 nm or more and 420 nm less.

The light absorbing material according to a 7th aspect of the present disclosure includes the nonlinear optical material according to any one of the 1st to the 6th aspects.

According to the 7th aspect, the light absorbing material has excellent two-photon absorption properties for light having a wavelength in a short wavelength region.

The recording medium according to an 8th aspect of the present disclosure includes:
a recording film containing the nonlinear optical material according to any one of the 1st to the 6th aspects.

According to the 8th aspect, the nonlinear optical material has excellent two-photon absorption properties for light having a wavelength in a short wavelength region. The recoding medium including a recording film containing such a nonlinear optical material is suitable for a recording medium that records information or reads out information.

The method for recording information according to a 9th aspect of the present disclosure includes:
preparing a light source emitting light having a wavelength of 390 nm or more and 420 nm or less; and
collecting the light from the light source and irradiating a recording region in a recording medium including the nonlinear optical material according to any one of the 1st to the 6th aspects with the light.

According to a 9th aspect, the nonlinear optical material has excellent two-photon absorption properties for light having a wavelength in a short wavelength region. According to the method for recording information using a recording medium including such a nonlinear optical material, it is possible to record information with a high recording density.

The method for reading out information according to a 10th aspect of the present disclosure is, for example, a method for reading out the information recorded by the recording method according to the 9th aspect and includes:
irradiating the recording region in the recording medium with light to measure an optical characteristic of the recording region; and
judging whether information is recorded in the recording region or not based on the optical characteristic.

In an 11th aspect of the present disclosure, for example, in the method for reading out information according to the 10th aspect, the optical characteristic may be the intensity of the light reflected in the recording region.

According to the 10th or the 11th aspect, it is easy to distinguish a recording region in which information is recorded.

Embodiments of the present disclosure will now be described with reference to the drawings. The present disclosure is not limited to the following embodiments.

A compound A of the present embodiment is represented by the following formula (1):

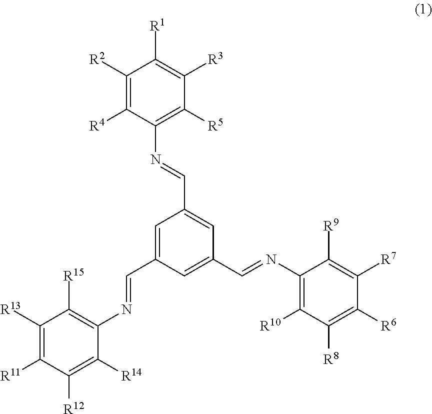

In the formula (1), R¹ to R¹⁵ each independently include at least one atom selected from the group consisting of H, C, N, O, F, P, S, Cl, I, and Br. R¹ to R¹⁵ may be each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

Examples of the halogen atom include F, Cl, Br, and I. In the present specification, a halogen atom may be called a halogen group.

The number of carbon atoms of the alkyl group is not particularly limited and is, for example, 1 or more and 20 or less. The number of carbon atoms of the alkyl group may be 1 or more and 10 or less or 1 or more and 5 or less from the viewpoint of easily synthesizing the compound A. The solubility of the compound A in a solvent or a resin composition can be adjusted by controlling the number of carbon atoms of the alkyl group. The alkyl group may be linear, branched, or cyclic. At least one of hydrogen atoms included in the alkyl group may be substituted with a group including at least one atom selected from the group consisting of N, O, P, and S. Examples of the alkyl group include a methyl group, an ethyl group, a propyl group, a butyl group, a 2-methylbutyl group, a pentyl group, a hexyl group, a 2,3-dimethylhexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a 2-methoxybutyl group, and a 6-methoxyhexyl group.

The halogenated alkyl group is a group in which at least one of hydrogen atoms included in the alkyl group is substituted with a halogen atom. The halogenated alkyl group may be a group in which all the hydrogen atoms included in the alkyl group are substituted with halogen atoms. Examples of the alkyl group include those mentioned above. A specific example of the halogenated alkyl group is —CF₃.

The unsaturated hydrocarbon group includes an unsaturated bond, such as a carbon-carbon double bond or a carbon-carbon triple bond. The number of the unsaturated bonds included in the unsaturated hydrocarbon group is, for example, 1 or more and 5 or less. The number of carbon atoms of the unsaturated hydrocarbon group is not particularly limited and is, for example, 2 or more and 20 or less and may be 2 or more and 10 or less or 2 or more and 5 or less. The unsaturated hydrocarbon group may be linear, branched, or cyclic. The at least one of hydrogen atoms included in the unsaturated hydrocarbon group may be substituted with a group including at least one atom selected from the group consisting of N, O, P, and S. Examples of the unsaturated hydrocarbon group include a vinyl group and an ethynyl group.

The hydroxyl group is represented by —OH. The carboxyl group is represented by —COOH. The alkoxycarbonyl group is represented by —COORₐ. The acyl group is represented by —COR_{b}. The amide group is represented by —CONR_{c}R_{d}. The nitrile group is represented by —CN. The alkoxy group is represented by —ORₑ. The acyloxy group is represented by —OCOR_{f}. The thiol group is represented by —SH. The alkylthio group is represented by —SR_{g}. The sulfonate group is represented by —SO₃H. The acylthio group is represented by —SCORₕ. The alkylsulfonyl group is represented by —SO₂Rᵢ. The sulfonamide group is represented by —SO₂NRⱼRₖ. The primary amino group is represented by —NH₂. The secondary amino group is represented by —NHRₗ. The tertiary amino group is represented by —NRₘRₙ. The nitro group is represented by —NO₂. Rₐ to Rₙ are each independently an alkyl group. Examples of the alkyl group include those mentioned above. However, R_{c} and R_{d} of the amide group and Rⱼ and Rₖ of the sulfonamide group may be each independently a hydrogen atom.

Specific examples of the alkoxycarbonyl group are —COOCH₃, —COO(CH₂)₃CH₃, and —COO(CH₂)₇CH₃. A specific example of the acyl group is —COCH₃. A specific example of the amide group is —CONH₂. Specific examples of the alkoxy group are a methoxy group, an ethoxy group, a 2-methoxyethoxy group, a butoxy group, a 2-methylbutoxy group, a 2-methoxybutoxy group, a 4-ethylthiobutoxy group, a pentyloxy group, a hexyloxy group, a heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a nonadecyloxy group, and an eicosyloxy group. A specific example of the acyloxy group is —OCOCH₃. A specific example of the acylthio group is —SCOCH₃. A specific example of the alkylsulfonyl group is —SO₂CH₃. A specific example of the sulfonamide group is —SO₂NH₂. A specific example of the tertiary amino group is —N(CH₃)₂.

However, in the formula (1), at least one selected from the group consisting of R¹ to R⁵, at least one selected from the group consisting of R⁶ to R¹⁰, and at least one selected from the group consisting of R¹¹ to R¹⁵ are represented by the following formula (2):

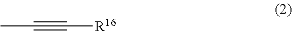

In the formula (2), R¹⁶ includes at least one atom selected from the group consisting of H, C, N, O, F, P, S, Cl, I, and Br. R¹⁶ may be a hydrogen atom or a substituent described in regarding R¹ to R¹⁵.

Incidentally, in the formula (1), R² to R⁵, R⁷ to R¹⁰, and R¹² to R¹⁵ may each have a small volume. In such a case, in R² to R⁵, R⁷ to R¹⁰, and R¹² to R¹⁵, steric hindrance is unlikely to occur. Accordingly, in the compound A, the flatness of the π electron conjugated system is improved, and the compound A therefore tends to have a large two-photon absorption cross section. R² to R⁵, R⁷ to R¹⁰, and R¹² to R¹⁵ may be each a hydrogen atom.

The compound A is, for example, a compound B represented by the following formula (3):

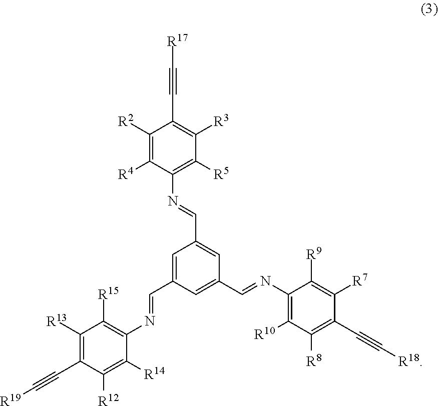

In the formula (3), R² to R⁵, R⁷ to R¹⁰, R¹² to R¹⁵, and R¹⁷ to R¹⁹ each independently include at least one atom selected from the group consisting of H, C, N, O, F, P, S, Cl, I, and Br. R² to R⁵, R⁷ to R¹⁰, R¹² to R¹⁵, and R¹⁷ to R¹⁹ are each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

Examples of the compound B represented by the formula (3) include a compound C represented by the following formula (5):

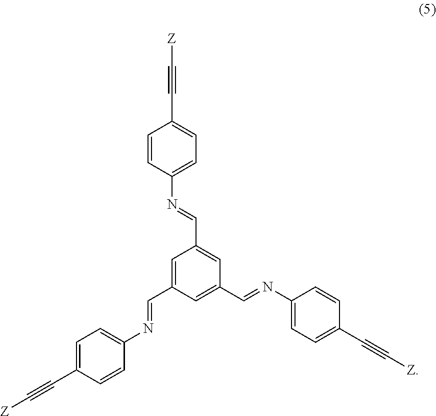

In the formula (5), three Zs are the same as each other and correspond to respectively R¹⁷ to R¹⁹ in the formula (3). In the formula (5), three Zs are, for example, hydrogen atoms.

The compound B may be a compound D represented by the following formula (4):

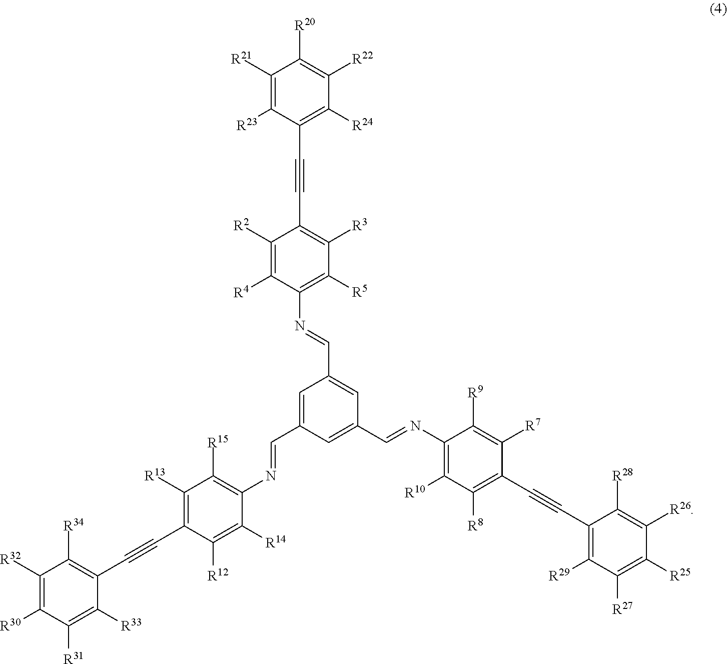

In the formula (4), R² to R⁵, R⁷ to R¹⁰, R¹² to R¹⁵, and R²⁰ to R³⁴ each independently include at least one atom selected from the group consisting of H, C, N, O, F, P, S, Cl, I, and Br. R² to R⁵, R⁷ to R¹⁰, R¹² to R¹⁵, and R²⁰ to R³⁴ are each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

At least one selected from the group consisting of R²⁰ to R²², R²⁵ to R²⁷, and R³⁰ to R³² may an electron donating group or an electron withdrawing group. Regarding R²⁰ to R²², R²⁵ to R²⁷, and R³⁰ to R³², the greater the electron donating property or the electron withdrawing property, the greater the deviation of electrons in the compound D. When the deviation of electrons in the compound D is large, electrons tend to largely move in the compound D when the compound D is excited. Such a compound D may have more excellent two-photon absorption properties. In other words, when at least one selected from the group consisting of R²⁰ to R²², R²⁵ to R²⁷, and R³⁰ to R³² is an electron donating group or an electron withdrawing group, the compound D may have a large two-photon absorption cross section.

The electron withdrawing group is, for example, a substituent having a positive substituent constant, σₚ value, in the Hammett formula. Examples of the electron withdrawing group include a halogen atom, a carboxyl group, a nitro group, a thiol group, a sulfonate group, an acyloxy group, an alkylthio group, an alkylsulfonyl group, a sulfonamide group, an acyl group, an acylthio group, an alkoxycarbonyl group, and a halogenated alkyl group.

The electron donating group is, for example, a substituent having a negative σₚ value. Examples of the electron donating group include an alkyl group, an alkoxy group, a hydroxyl group, and an amino group.

Each of R²³, R²⁴, R²⁸, R²⁹, R³³, and R³⁴ may have a small volume. In such a case, in R²³, R²⁴, R²⁸, R²⁹, R³³, and R³⁴, steric hindrance is unlikely to occur. Accordingly, in the compound D, the flatness of the π electron conjugated system is improved. When the π electron conjugated system of the compound D has high flatness, the compound D tends to have a large two-photon absorption cross section. R²³, R²⁴, R²⁸, R²⁹, R³³, and R³⁴ may be each a hydrogen atom.

Examples of the compound D represented by the formula (4) include a compound E represented by the following formula (6):

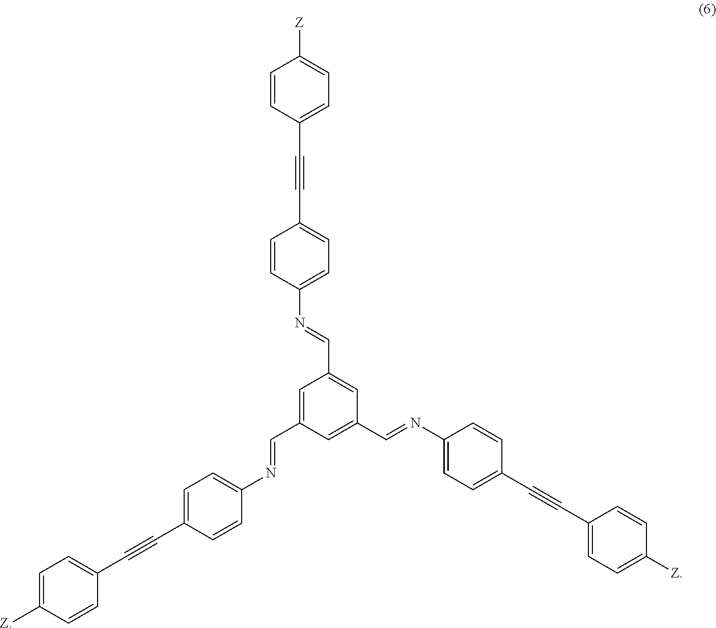

In the formula (6), three Zs are the same as each other and correspond to respectively R²⁰, R²⁵, and R³⁰ in the formula (4). Examples of Z in the formula (6) are shown in Table 1. In the formula (6), three Zs are, for example, hydrogen atoms.

TABLE 1

| | Z |
|---|---|
| 1 | —H |
| 2 | —F |
| 3 | —CH₃ |
| 4 | —C₂H₅ |
| 5 | —CF₃ |
| 6 | —OH |
| 7 | —COOH |
| 8 | —COOCH₃ |
| 9 | —COOC₄H₉ |
| 10 | —COOC₈H₁₇ |
| 11 | —COCH₃ |
| 12 | —CONH₂ |
| 13 | —CN |
| 14 | —OCH₃ |
| 15 | —OCOCH₃ |
| 16 | —SH |
| 17 | —SO₃H |
| 18 | —SCOCH₃ |
| 19 | —SO₂CH₃ |
| 20 | —SO₂NH₂ |
| 21 | —NH₂ |
| 22 | —N(CH₃)₂ |
| 23 | —NO₂ |
| 24 | —C(CH₃)₃ |

The compound C represented by the formula (5) and the compound E represented by the formula (6) may be synthesized by any method. The compounds C and E can be synthesized by, for example, the following method. First, a compound F represented by the following formula (7) is prepared. The compound F is benzene-1,3,5-trialdehyde.

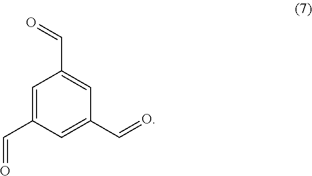 [SMILES: O=Cc1cc(C=O)cc(C=O)c1]

Subsequently, the compound F is subjected to a dehydration condensation reaction with a compound G including an amino group. Consequently, the compound C or E can be synthesized. The structure of the compound G is determined depending on the structure of a target compound. The conditions of the dehydration condensation reaction can be appropriately adjusted, for example, depending on the compounds F and G.

The compound A represented by the formula (1) has excellent two-photon absorption properties for light having a wavelength in a short wavelength region. As an example, when the compound A is irradiated with light having a wavelength of 405 nm, two-photon absorption significantly occurs in the compound A.

The two-photon absorption cross section of the compound A for light having a wavelength of 405 nm may be 25000 GM or more, 30000 GM or more, 40000 GM or more, or 50000 GM or more. The upper limit of the two-photon absorption cross section of the compound A is not particularly limited and is, for example, 100000 GM. The two-photon absorption cross section can be measured by, for example, the Z-scan method described in J. Opt. Soc. Am. B, 2003, Vol. 20, p. 529. The Z-scan method is widely used as a method for measuring a nonlinear optical constant. In the Z-scan method, a measurement sample is moved along the irradiation direction of laser beams at near the focal point where the beams are collected. At this time, a change in the amount of light transmitted through the measurement sample is recorded. In the Z-scan method, the power density of incident light changes according to the position of the measurement sample, Accordingly, when the measurement sample performs nonlinear absorption, the amount of transmitted light decreases when the measurement sample is located at near the focal point of laser beams. The two-photon absorption cross section can be calculated by fitting the change in the amount of transmitted light to the theoretical curve estimated from, for example, the intensity of incident light, the thickness of the measurement sample, and the concentration of the compound A in the measurement sample.

When the compound A performs two-photon absorption, the compound A absorbs about twice as much energy as that of the light irradiated to the compound A. The wavelength of light having about twice the energy of the light having a wavelength of 405 nm is, for example, 200 nm. That is, when the compound A is irradiated with light having a wavelength of approximately 200 nm, one-photon absorption may be caused in the compound A. Furthermore, in the compound A, one-photon absorption may occur for light having a wavelength near the wavelength region causing two-photon absorption.

The compound A represented by the formula (1) can be used as, for example, a component of a light absorbing material. That is, from another aspect, the present disclosure provides a light absorbing material including the compound A represented by the formula (1). The light absorbing material includes, for example, the compound A as a main component. The term “main component” means a component included most in the light absorbing material in terms of weight ratio. For example, the light absorbing material consists essentially of the compound A. The term “consist essentially of” means that other components that change the essential characteristics of the mentioned material are eliminated. However, the light absorbing material may include impurities in addition to the compound A. The light absorbing material functions as, for example, a multi-photon absorption material, such as a two-photon absorption material. In particular, the light absorbing material including the compound A has excellent two-photon absorption properties for light having a wavelength in a short wavelength region.

The compound A is used in, for example, a device using light having a wavelength in a short wavelength region. That is, from another aspect thereof, the present disclosure is used in a device using light having a wavelength of 390 nm or more and 420 nm or less and provides a compound A represented by the formula (1). Examples of such a device include a recording medium, a molding machine, and a fluorescence microscope. Examples of the recording medium include a three-dimensional optical memory. A specific example of the three-dimensional optical memory is a three-dimensional optical disk. Examples of the molding machine include a stereolithography apparatus, such as a 3D printer. Examples of the fluorescence microscope include two-photon fluorescence microscope. The light that can be used in these devices has, for example, a high photon density at near the focal point thereof. The power density at near the focal point of the light that is used in each of the devices is, for example, 0.1 W/cm² or more and 1.0×10²⁰ W/cm² or less. This power density at near the focal point of light may be 1.0 W/cm² or more, 1.0×10² W/cm² or more, or 1.0×10⁵ W/cm² or more. As the light source of the device, for example, a femtosecond laser, such as a titanium sapphire laser, or a pulse laser having a pulse width of picoseconds to nanoseconds, such as a semiconductor laser, can be used.

The recording medium includes a thin film called, for example, a recording layer or a recording film. In the recording medium, information is recorded in the recording layer or the recording film. As an example, the thin film as a recording layer or a recording film contains the compound A. That is, from another respect thereof, the present disclosure provides a recording medium including a recording film containing the compound A represented by the formula (1).

The recording layer may further include a high molecular compound functioning as a binder, in addition to the compound A. The recording medium may include a dielectric layer in addition to the recording layer. The recording medium includes, for example, a plurality of recording layers and a plurality of dielectric layers. In the recording medium, a plurality of the recording layers and a plurality of the dielectric layers may be alternately stacked.

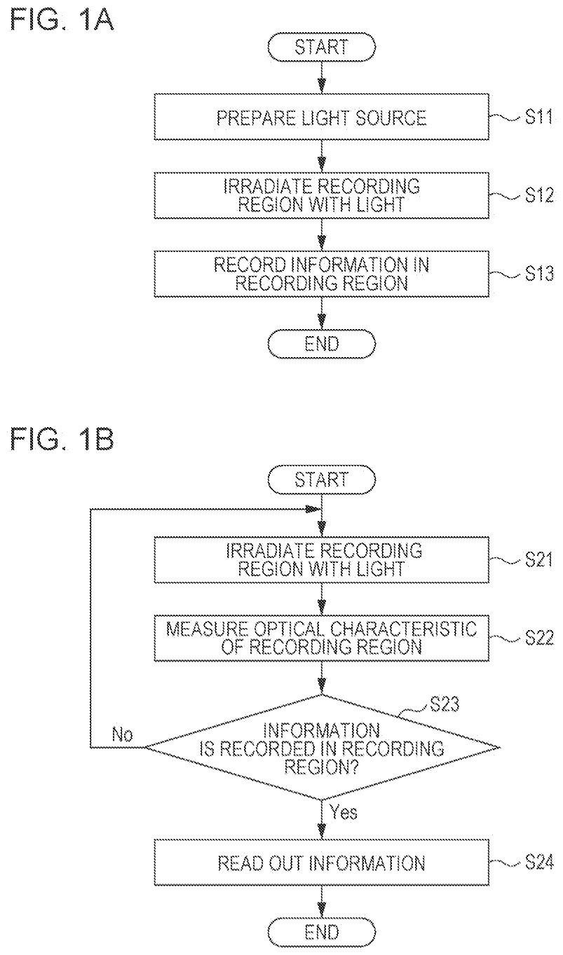

Subsequently, a method for recording information using the recording medium will be described. FIG. 1A is a flow chart relating to a method for recording information using the above-described recording medium. First, in step S11, a light source emitting light having a wavelength of 390 nm or more and 420 nm or less is prepared. As the light source, for example, a femtosecond laser, such as a titanium sapphire laser, can be used. Alternatively, as the light source, a pulse laser having a pulse width of picoseconds to nanoseconds, such as a semiconductor laser, may be used. Subsequently, in step S12, light from the light source is collected with, for example, a lens, and the recording region in the recording medium is irradiated with the light. The power density of this light at near the focal point is, for example, 0.1 W/cm² or more and 1.0×10²⁰ W/cm² or less. The power density of this light at near the focal point may be 1.0 W/cm² or more, 1.0×10² W/cm² or more, or 1.0×10⁵ W/cm² or more. In the present specification, the recording region is a spot that is present in the recording layer and can record information by being irradiated with light.

In the recording region irradiated with the light, a physical change or a chemical change occurs. For example, when the compound A absorbed light returns from the transition state to the ground state, heat is generated. The binder present in the recording region is changed in quality by this heat. Consequently, the optical characteristics of the recording region change. For example, the intensity of light reflected in the recording region, the reflective index of light at the recording region, the absorption of light in the recording region, and the refractive index of light at the recording region change. In the recording region irradiated with light, the intensity or the wavelength of fluorescence light emitted from the recording region may change. Consequently, the information can be recorded in the recording region (step S13).

Then, a method for reading out information using the above-described recording medium will be described. FIG. 1B is a flow chart relating to a method for reading out information using the above-described recording medium. First, in step S21, the recording region in the recording medium is irradiated with light. The light used in step S21 may be the same as or different from the light used for recording information in the recording medium. Subsequently, in step S22, an optical characteristic of the recording region is measured. In step S22, for example, as the optical characteristic of the recording region, the intensity of light reflected in the recording region is measured. In step S22, as the optical characteristic of the recording region, for example, the reflective index of light at the recording region, the absorption of light in the recording region, the refractive index of light at the recording region, or the intensity or the wavelength of fluorescence light emitted from the recording region may be measured.

Subsequently, in step S23, whether information is recorded in the recording region or not is judged based on the optical characteristic of the recording region. For example, when the intensity of light reflected in the recording region is less than or equal to a specific value, it is judged that information is recorded in the recording region. In contrast, when the intensity of light reflected in the recording region is higher than the specific value, it is judged that information is not recorded in the recording region. When it is judged that information is not recorded in the recording region, the process returns step S21, and the same procedure is performed for another recording region. When it is judged that information is recorded in the recording region, in step S24, information is read out.

The methods for recording and reading out information using the above-described recording medium can be performed with, for example, a known recording apparatus. The recording apparatus includes, for example, a light source for irradiating the recording region in the recording medium with light, a measuring instrument for measuring an optical characteristic of the recording region, and a controller for controlling the light source and the measuring instrument.

The molding machine performs molding by, for example, irradiating a photocurable resin composition with light to cure the resin composition. As an example, the photocurable resin composition for stereolithography includes the compound A. The photocurable resin composition includes, for example, a polymerizable compound and a polymerization initiator, in addition to the compound A. The photocurable resin composition may further include an additive, such as a binder resin. The photocurable resin composition may include an epoxy resin.

In the fluorescence microscope, for example, a biological sample including a fluorescent dye material is irradiated with light, and the fluorescence emitted from the dye material can be observed. As an example, the fluorescent dye material to be added to a biological sample contains the compound A.

### EXAMPLES

The present disclosure will now be described by Examples in further detail. Incidentally, the following Examples are merely examples, and the present disclosure is not limited to the following Examples.

### Example 1

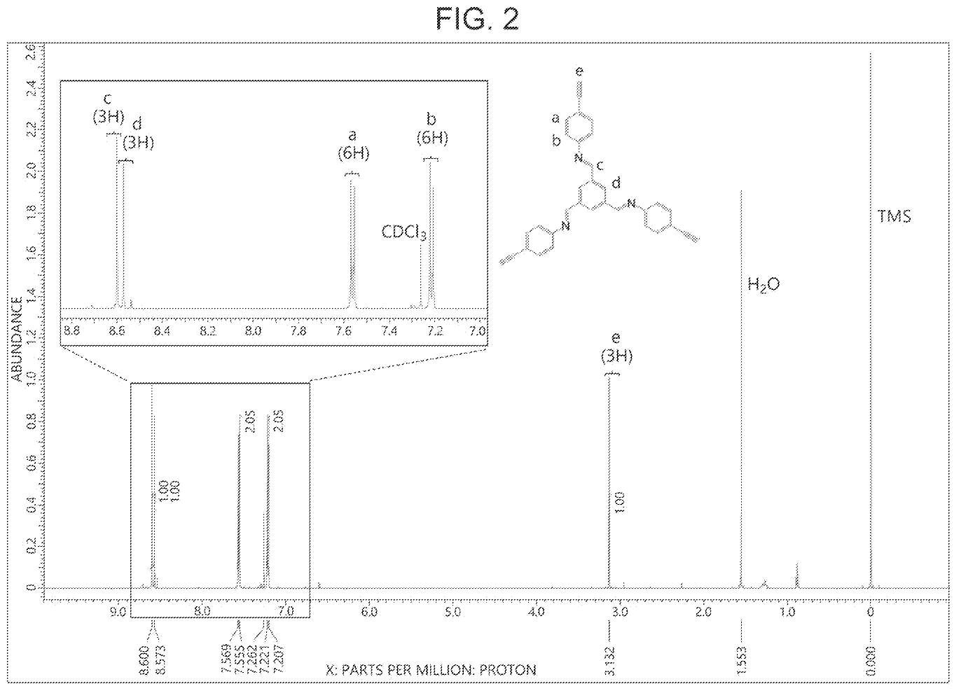

First, a 100-mL eggplant shaped flask was charged with benzene-1,3,5-trialdehyde (manufactured by Sigma-Aldrich Co. LLC) and 4-ethynylaniline (manufactured by Tokyo Chemical Industry Co., Ltd.) as raw materials and ethanol (manufactured by FUJIFILM Wako Pure Chemical Corporation) as a solvent, and the raw materials were dissolved in the solvent. Subsequently, the resulting solution was heated to reflux for 12 hours while stirring with a stirrer. Consequently, a reaction product was generated. Subsequently, this reaction product was subjected to solid-liquid separation. The resulting solid was vacuum-dried to obtain a compound of Example 1. The compound of Example 1 corresponds to the compound C represented by the above-described formula (5) in which three Zs are hydrogen atoms. The compound of Example 1 was identified by ¹H-NMR. FIG. 2 is a graph showing a ¹H-NMR spectrum of the compound of Example 1. The ¹H-NMR spectrum of the compound of Example 1 is as follows.

### ¹H-NMR (600 MHz, DMSO-D6) δ7.21-7.26 (m, 6H), 7.56 (d, J=8.4 Hz, 6H), 8.57 (s, 3H), 8.60 (s, 3H).

### Example 2

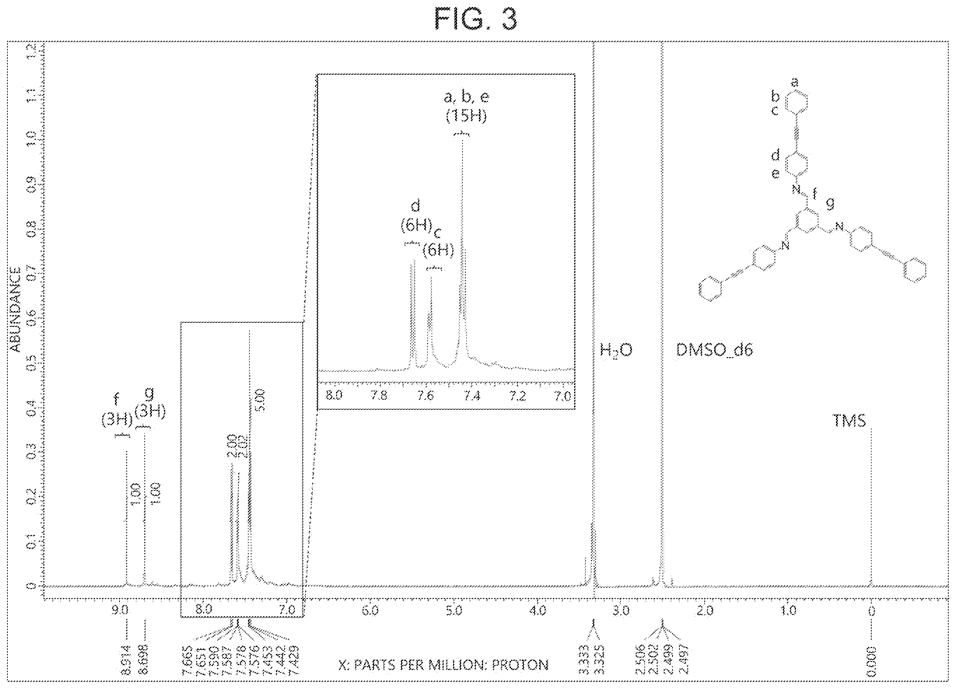

First, a 100-mL eggplant shaped flask was charged with benzene-1,3,5-trialdehyde (manufactured by Sigma-Aldrich Co. LLC) and 4-phenylethynyl phenylamine (manufactured by BLD Pharmatech Ltd.) as raw materials and ethanol (manufactured by FUJIFILM Wako Pure Chemical Corporation) as a solvent, and the raw materials were dissolved in the solvent. Subsequently, the resulting solution was heated to reflux for 12 hours while stirring with a stirrer. Consequently, a reaction product was generated. Subsequently, this reaction product was subjected to solid-liquid separation. The resulting solid was vacuum-dried to obtain a compound of Example 2. The compound of Example 2 corresponds to the compound E represented by the above-described formula (6) in which three Zs are hydrogen atoms. The compound of Example 2 was identified by ¹H-NMR. FIG. 3 is a graph showing a ¹H-NMR spectrum of the compound of Example 2. The ¹H-NMR spectrum of the compound of Example 2 is as follows.

### ¹H-NMR (600 MHz, DMSO-D6) δ7.43-7.45 (m, 15H), 7.58-7.59 (m, 6H), 7.66 (d, J=8.4 Hz, 6H), 8.70 (s, 3H), 8.91 (s, 3H).

### Comparative Examples 1 and 2

As a compound of Comparative Example 1, hexakis(phenylethynyl)benzene was prepared. As a compound of Comparative Example 2, 1,2,4,5-tetrakis(phenylethynyl)benzene was prepared. The compound of Comparative Example 1 is a compound having a largest two-photon absorption cross section for light having a wavelength of 405 nm, among the compounds that have been reported in the field of two-photon absorption material.

### Measurement of Two-photon Absorption Cross Section

The compounds of Examples and Comparative Examples were measured for the two-photon absorption cross sections for light having a wavelength of 405 nm. The two-photon absorption cross section was measured using the Z scan method described in J. Opt. Soc. Am. B, 2003, Vol. 20, p. 529. As the light source for measuring the two-photon absorption cross section, a titanium sapphire pulse laser was used. For the details, the sample was irradiated with the second high-frequency of the titanium sapphire pulse laser. The pulse width of the laser was 80 fs. The repetition frequency of the laser was 1 kHz. The average power of the laser was changed within a range of 0.01 mW or more and 0.08 mW or less. The light from the laser was light having a wavelength of 405 nm. For the details, the light from the laser had a central wavelength of 402 nm or more and 404 nm or less. The full width at half maximum of light from the laser was 4 nm. The results are shown in Table 2.

TABLE 2

| | Two-photon absorption |
|---|---|
| | cross section (GM) |
| Example 1 | 55602 |
| Example 2 | 30275 |
| Comparative Example 1 | 23000 |
| Comparative Example 2 | 5400 |

As shown in Table 2, in both the compounds of Examples 1 and 2 corresponding to the compound A represented by the formula (1), the two-photon absorption cross sections for light having a wavelength of 405 nm were above 30000 GM. As described above, the compound of Comparative Example 1 is a compound having a largest two-photon absorption cross section for light having a wavelength of 405 nm among the compounds that have been reported in the field of two-photon absorption material. Compared to this compound of Comparative Example 1, the compounds of Examples 1 and 2 had very large two-photon absorption cross sections. It is revealed from this result that the compound A represented by the formula (1) has excellent two-photon absorption properties for light having a wavelength in a short wavelength region. The compound A represented by the formula (1) is a 3-substituted benzene having an enlarged π electron conjugated system and has an imine group in the molecular framework. It is inferred that the compound A has excellent two-photon absorption properties resulting from such a structure.

The nonlinear optical material of the present disclosure can be utilized in uses, such as a recording layer of a three-dimensional optical memory and a photocurable resin composition for stereolithography. The nonlinear optical material of the present disclosure tends to have excellent two-photon absorption properties for light having a wavelength in a short wavelength region. Accordingly, the nonlinear optical material of the present disclosure can realize extremely high spatial resolution in uses such as a three-dimensional optical memory and a molding machine. According to the nonlinear optical material of the present disclosure, it is possible to perform two-photon absorption by laser light of a small light intensity, compared to the nonlinear optical materials that have been reported in the field of two-photon absorption material.

What is claimed is:

1. A nonlinear optical material represented by a following formula (1): 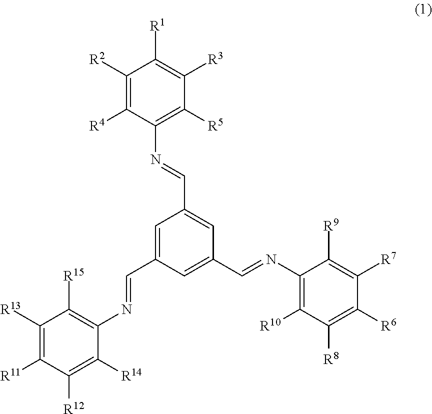
in the formula (1), R¹ to R¹⁵ each independently include at least one atom selected from the group consisting of H, C, N, O, F, P, S, Cl, I, and Br, wherein
at least one selected from the group consisting of R¹ to R⁵, at least one selected from the group consisting of R⁶ to R¹⁰, and at least one selected from the group consisting of R¹¹ to R¹⁵ are represented by a following formula (2): 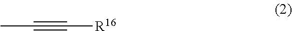
in the formula (2), R¹⁶ includes at least one atom selected from the group consisting of H, C, N, O, F, P, S, Cl, I, and Br.

2. The nonlinear optical material according to claim 1, wherein
the nonlinear optical material is represented by a following formula (3): 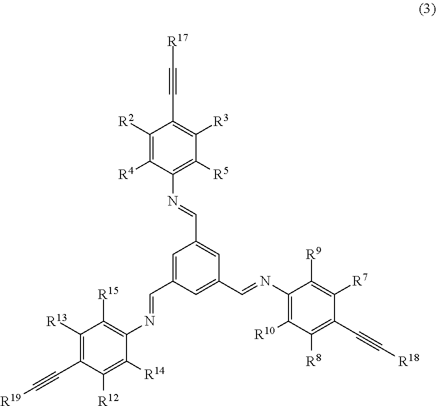
in the formula (3), R² to R⁵, R⁷ to R¹⁰, R¹² to R¹⁵, and R¹⁷ to R¹⁹ each independently include at least one atom selected from the group consisting of H, C, N, O, F, P, S, Cl, I, and Br.

3. The nonlinear optical material according to claim 2, wherein
the R² to the R⁵, the R⁷ to the R¹⁰, the R¹² to the R¹⁵, and the R¹⁷ to the R¹⁹ are each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

4. The nonlinear optical material according to claim 1, wherein
the nonlinear optical material is represented by a following formula (4): 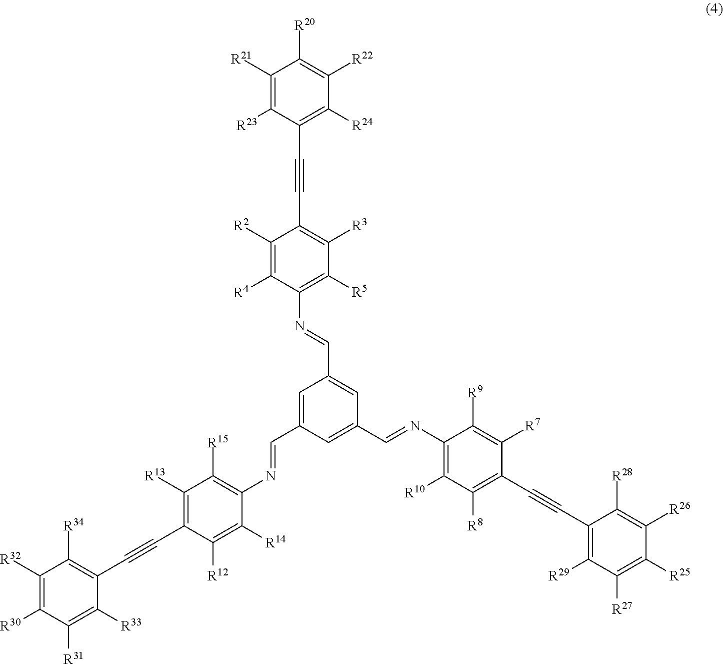
in the formula (4), R² to R⁵, R⁷ to R¹⁰, R¹² to R¹⁵, and R²⁰ to R³⁴ each independently include at least one atom selected from the group consisting of H, C, N, O, F, P, S, Cl, I, and Br.

5. The nonlinear optical material according to claim 4, wherein
the R² to the R⁵, the R⁷ to the R¹⁰, the R¹² to the R¹⁵, and the R²⁰ to the R³⁴ are each independently a hydrogen atom, a halogen atom, an alkyl group, a halogenated alkyl group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

6. The nonlinear optical material according to claim 1, wherein
the nonlinear optical material is used in a device using light having a wavelength of 390 nm or more and 420 nm or less.

7. A light absorbing material comprising the nonlinear optical material according to claim 1.

8. A recording medium comprising a recording film including the nonlinear optical material according to claim 1.

9. A method for recording information, comprising:
preparing a light source emitting light having a wavelength of 390 nm or more and 420 nm or less; and
collecting the light from the light source and irradiating a recording region in a recording medium including the nonlinear optical material according to claim 1 with the light.

10. A method for reading out information recorded by the method according to claim 9, comprising:
irradiating the recording region in the recording medium with light to measure an optical characteristic of the recording region; and
judging whether information is recorded in the recording region or not based on the optical characteristic.

11. The method according to claim 10, wherein
the optical characteristic is an intensity of the light reflected in the recording region.
